(19) United States
(12) Patent Application Publication
(10) Pub. No.: US 2025/0235552 A1
(43) Pub. Date: Jul. 24, 2025

(54) INTEIN TECHNOLOGY TO DELIVER ABCA3 LIPID TRANSPORTER FOR ABCA3 DEFICIENCY

(71) Applicant: Children's Hospital Medical Center, Cincinnati, OH (US)

(72) Inventors: Yutaka Maeda, Cincinnati, OH (US); William Douglas Stuart, Cincinnati, OH (US); Iris Fink-Baldauf, Cincinnati, OH (US)

(21) Appl. No.: 18/415,949

(22) Filed: Jan. 18, 2024

(51) Int. Cl. A61K 48/00; C12N 9/22; C12N 15/11

(52) U.S. Cl. CPC A61K 48/005; C12N 9/22; C12N 15/11; C12N 2310/20

(57) ABSTRACT

In order to develop a therapeutic strategy to rescue ABCA3 deficiency caused by a splicing mutation of ABCA3, we have designed an experimental scheme using CRISPR/Cas9 to delete a loxP site located at an intronic region of ABCA3 flox mice (Sftpc-Cre;Abca3flox/flox). The deletion of this loxP site extended survival of the mice by only a week, probably due to insufficient re-ligation efficiency after the CRISPR/Cas9 deletion. An alternative approach to rescuing ABCA3 deficiency caused by mutations in ABCA3, including coding or non-coding (e.g., intronic) mutations, is to deliver intact ABCA3 into lung alveolar type II cells using AAV (e.g., AAV6.2FF), a clinically approved delivery system. Due to the large size of ABCA3 (1704 amino acids), it is technically impossible to package full length ABCA3 cDNA into an AAV virus. However, a recent technological advance using intein makes it possible to split ABCA3 cDNA into two fragments for separate AAV delivery to the cells wherein the translated ABCA3 protein fragments would recombine into full length ABCA3 protein.

### STATEMENT REGARDING FEDERALLY SPONSORED RESEARCH OR DEVELOPMENT

This invention was made with government support under HL134745 awarded by the National Institutes of Health. The government has certain rights in the invention.

### DESCRIPTION

ABCA3: In order to develop a therapeutic strategy to rescue ABCA3 deficiency caused by a splicing mutation of ABCA3, we have designed an experimental scheme using CRISPR/Cas9 to delete a loxP site located at an intronic region of ABCA3 flox mice (Sftpc-Cre;Abca3flox/flox). The deletion of this loxP site extended survival of the mice by only a week, probably due to insufficient re-ligation efficiency after the CRISPR/Cas9 deletion. An alternative approach to rescuing ABCA3 deficiency caused by mutations in ABCA3, including coding or non-coding (e.g., intronic) mutations, is to deliver intact ABCA3 into lung alveolar type II cells using AAV (e.g., AAV6.2FF), a clinically approved delivery system. Due to the large size of ABCA3 (1704 amino acids), it is technically impossible to package full length ABCA3 cDNA into an AAV virus. However, a recent technological advance using intein makes it possible to split ABCA3 cDNA into two fragments for separate AAV delivery to the cells wherein the translated ABCA3 protein fragments would recombine into full length ABCA3 protein. The technology using intein has been successfully used in in vivo CRISPR/Cas9 translational studies using AAV. In the past year, we attached intein-N terminus and intein-C terminus fragments with N-terminus ABCA3 and C-terminus ABCA3 fragments, respectively, and successfully produced recombined ABCA3 protein in 293T cells in vitro. We have also succeeded in producing AAV6.2FF virus carrying N-ABCA3-intein-N and C-ABCA3-intein-C fragment, separately. Using these AAV viruses, we plan to assess whether we can rescue our ABCA3 deficient model mice. We anticipate that we will be able to further extend the survival rate of ABCA3 deficient mice.

### PUBLICATIONS

Stuart WD, Fink-Baldauf IM, Tomoshige K, Guo M, Maeda Y. CRISPRi-mediated functional analysis of NKX2-1-binding sites in the lung. Commun Biol. 2021 May 12;4 (1): 568. doi: 10.1038/s42003-021-02083-4. PMID: 33980985; PMCID: PMC8115294.

Inventor: Yutaka Maeda, William Stuart, Iris Fink-Baldauf

Assignee: Cincinnati Children's Hospital Medical Center

What is claimed:

1. A method for recuing ABCA3 deficiency caused by a splicing mutation of ABACA3, comprising using CRISPR/Cas9 to delete a loxP site located at an intronic region of ABCA3 flox.
